(19) United States
(12) Patent Application Publication
(10) Pub. No.: US 2025/0325310 A1
(43) Pub. Date: Oct. 23, 2025

(54) ADJUSTABLE PEDIATRIC HIP SCREW SYSTEM

(71) Applicants: Mahmoud Smida, Tunis (TN); IMed Zavani, Dollars-Des-Ormeaux (CA)

(72) Inventors: Mahmoud Smida, Tunis (TN); IMed Zavani, Dollars-Des-Ormeaux (CA)

(73) Assignee: Imed Inc, ST LEONARD (CA)

(21) Appl. No.: 18/640,029

(22) Filed: Apr. 19, 2024

(51) Int. Cl. A61B 17/74; A61B 17/68; A61B 17/88

(52) U.S. Cl. CPC A61B 17/742; A61B 17/8875; A61B 2017/681

(57) ABSTRACT

An adjustable pediatric hip screw system designed for easy intra-operative length adjustment using a specialized inserter. The system ensures bone stimulation through the integration of a spring positioned between the end cap and the male component. Additionally, the screw's unique geometry effectively hinders cell migration through the physis. The female component is equipped with a dual flat-shaped shaft, which can be achieved either internally or externally through specific manufacturing processes. The male component is engineered to match the form of the female component, featuring a double flat to prevent rotation and a distal feature facilitating insertion into the femoral head.

### TECHNICAL FIELD

The present document generally relates to an adjustable pediatric hip screw. The system screw presented in this patent consists of an adjustable inserter, a male component, and a female component. The screw is presented in one single size that can adjusted to any femoral neck length to treat various pediatric bone diseases.

### BACKGROUND

The Slipped capital femoral epiphysis (SCFE) is the most common hip disease in adolescents, affecting approximately 1 in 10 per 100,000 (Larson, et al. 2010). SCFE is characterized by a slippage of the capital femoral epiphysis inferiorly and posteriorly to the femoral neck on a pathologically affected sub-capital growth plate (Millis and Novais 2011).

Without stabilization, the epiphysis continues to slip, exacerbating the condition and raising the risk of developing coxarthrosis through cervical-acetabular impingement (Oduwole, et al. 2017) Treatment for SCFE is focused on achieving permanent stability through surgical fixation of the capital epiphysis to the femoral neck bone, thereby halting further slippage.

The “Gold Standard” of treatment involves in-situ fixation of the epiphysis (Aronson and Carlson 1992;) (Gonzalez, et al. 1998). This approach is founded on the understanding that the sub-capital growth plate is central to the disease pathology, and halting its growth is deemed the safest course of action (Early, et al. 2011). Typically, in this “Gold Standard” treatment, in-situ fixation is achieved using by pinning either a threaded percutaneous screw or multiple K-wires. In both methods, the hardware is inserted through the sub-capital growth cartilage to induce epiphysiodesis (Peck K 2014).

Numerous studies have demonstrated that this technique effectively halts the progression of slippage and carries a low risk of complications (Rostoucher, et al. 1996). Fixation with multiple K-wires seems to affect the growth plate and the remodeling capabilities of the proximal femur less adversely (Seller, et al. 2006). However, it's worth noting that pins often dislodge from the epiphysis, causing intra-articular irritation. Additionally, repeated pinning is frequently necessary (Sailhan F. 2011) and the overall incidence of these complications appears to correlate with the number of pins inserted (Seller, et al. 2006).

In Scandinavia, in-situ fixation with the Hansson nail, a smooth, non-threaded nail with a hook, has been successfully used for decades (Hägglund G 1987)

In France, a cannulated screw with a proximal thread has been developed to stabilize the epiphysis using its distal smooth part (Sailhan F. 2011)

In Germany, the DET-screw has been utilized in numerous surgeries with successful outcomes, as summarized by (Schumann E., 2016). The study that was conducted between 2006 and 2014 confirmed that the use of telescopic screws is a safe procedure for both affected and non-affected hips. This approach serves as an adequate substitute for the commonly used method of pinning with K-wires.

In 2021, (Morash K 2021) presented a retrospective study on free gliding screws introduced to the market by Pega Medical in 2013. The study demonstrated that when treating SCFE with an FG screw, femoral neck cam deformity remodels more effectively. Additionally, the authors found that telescopic screws allow ongoing growth of prophylactically treated hips, while standard screws promote coxa breva.

US20100036440A1describes a collapsible bone screw apparatus, comprised of a male and female component with two threaded ends, being telescopically received, that is self-tapping and adjustable with bone growth. The two threaded components may have matching or similar pitches. The diameter of the threaded head of the female component is substantially larger than the diameter of the smooth shaft, which may cause epiphysiodesis and may slow significantly the residual bone growth. This makes the invention use similar to a standard cannulated screw. Besides the inventor provided a variety of sizes to accommodate the child's bone anatomy. The female component is inserted initially which requires the removal of a higher volume of bone. This may change drastically the femoral neck natural architecture. The male component head is buried beyond the lateral cortex of the femur which may cause positive migration of the assembled screw. The male component has no end cap which may create an inner bone growth that may lead to difficult hardware removal and may cause damage to the child's bone.

U.S. Pat. No. 9,814,501 B Describes An evolved version of Morris et al. screw assembly, where the major change is the form of the trilobe that was added to differentiate the two screws. In this description, the diameter of the threaded head of the female component is equal to the diameter of the smooth shaft, which may cause epiphysiodesis and may reduce residual bone growth. The invention provides dissimilar sizes to accommodate the size of the child's bone. The surgeon has to carry an inventory of all possible sizes and figure the best fit. This option of a full tray inventory is not possible for all hospitals or clinical centers due to high cost. Besides, Pre-operative planning is required to ensure proper screw size selection. So, the surgeon has to carry an accurate surgery plan for each of the cases. This may lead to various problems related to planning, ordering, and revising the surgery. The female component is inserted initially which requires the removal of a higher volume of bone. This may change drastically the femoral neck natural architecture, the bone integrity and the healing delay.

U.S. Pat. No. 8,702,768 B2 describes a cannulated bone screw system that includes being telescopically received, self-tapping, and capable of adjusting with bone growth. The device utilizes a spring and has only one distal threaded end. The female component has self-tapping features that allow the insertion of the screw. However, these features do not allow the female component to be properly fixed in the cortical bone and may be loosened easily. In fact, there is no bone purchase between the screw and the cortical bone. The female component may be loosened easily due to micro-motions and early weight bearing. In this case a negative migration of the screw may occur damaging soft tissues and blocking the physis growth. Also, the diameter of the threaded head of the female component is substantially larger than the diameter of the smooth shaft, which may cause epiphysiodesis and may decrease significantly the residual bone growth. This makes the invention similar to a standard cannulated screw, but not recommended for pediatric applications. The insertion of such a device in a child bone may change drastically the femoral neck natural architecture. The invention has a spring that creates a compression force on the proximal thread as this thread goes through the growth plate. For pediatric applications a compression force on the growth plate may cause a growth stoppage. This invention has to be limited to adult applications only due to the describes reasons above. Finally, the male component has no end cap to reduce the risk of inner bone growth.

US 2012/0184993 A1 describes a description of device for securely attaching bones together and, more specifically relates to a facet screw having features that facilitate improved relative attachment of vertebra through the facets. The presented device encompasses two component a threaded male and a threaded female, wherein the female component is expandable. The expansion of the females allows a better fixation of the device to the bone. Nevertheless, as presented this device cannot grow with the bone and have no gliding effect.

WO2020005971A1 describes a Length adjustable modular screw system where the female component is fully threaded and the male component is not threaded. The system is rotationally stable using a locking mechanism using lateral set screws. The length adjustment is performed by the mean of two set screws lodged on the female components and sliding above the male components. The device does not allow telescoping as once the length is fixed it cannot glide when bone growth occurs.

These surgical options and the cited inventions have specific drawbacks, including the need to use implants left protruding at the insertion site or changing drastically the architecture of the bone. The current invention is a new variant of telescopic screw with improvement and new features coming from all the performed SCFE surgeries by Dr. Mahmoud Smida.

### SUMMARY

A female component with a threaded shaft having a double flat.

A female component with a threaded cannulated shaft.

A female component with a head having a cross shape for screw driving.

A female component with two self-tapping features.

An end cap that can be tightened to the head of the female

A stimulation spring is nested between the male component and the cap.

A male component with a head partially threaded and with two self-tap features

A male component with a shaft having two flats.

A male component with an overhead smooth part larger than the thread.

An adjustment and insertion instrument with a threaded shaft

An adjustment and insertion instrument with a nut that controls the final length of the screw

An adjustment and insertion instrument with a connector is connected to the nut and is intended to drive the screw to desired position.

Additional objectives, advantages, and novel features will be set forth in the description which follows or will become apparent to those skilled in the art upon examination of the drawings and detailed description which follows.

### BRIEF DESCRIPTION OF THE DRAWINGS

FIG. 1 is a side view of the Pediatric Hip Screw Assembly

FIG. 2 is a section view of the Pediatric Hip Screw Assembly

FIG. 3 is a side, isometric and section views of the female component

FIG. 4 is a side view of the male component

FIG. 5 is an isometric view of the adjustable inserter

FIG. 6 is an Isometric view of the adjustable driver and the screw

Reference characters indicate corresponding elements in the views of the drawings. The titles used in the figures should not be interpreted as limiting the scope of the claims

### DETAILED DESCRIPTION

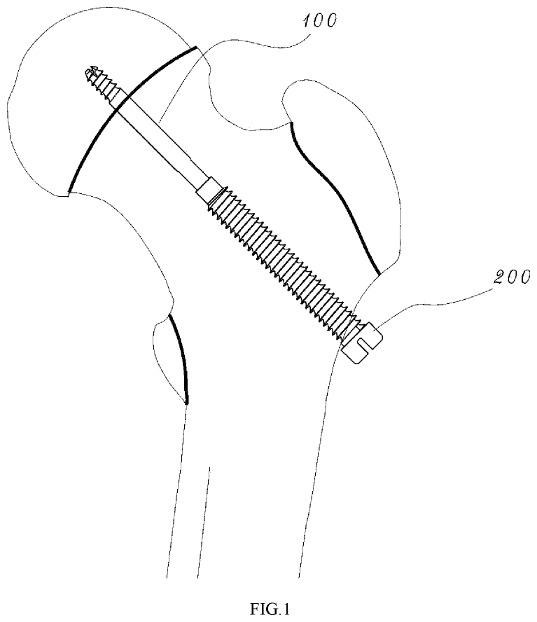

Referring to FIG. 1, the screw assembly is shown in a side view being inserted in the femoral neck with SCFE disease. The view illustrates the female component 200 fixed to the cortex of the lateral bone and along the femoral neck. The male component 100, is fixed above the physis and along the femoral head.

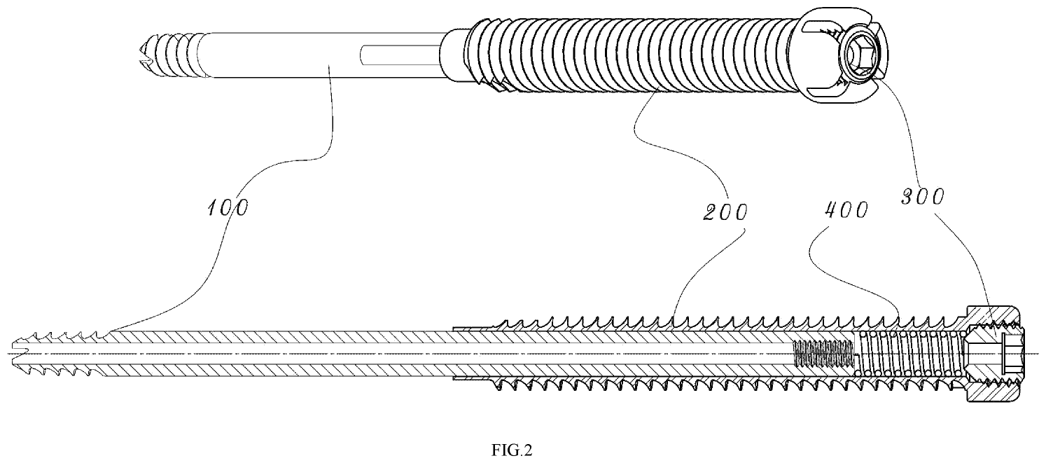

Referring FIG. 2 presents an isometric view of the screw with a section view showing the three implantable components: the male component 100, the female component 200, the end cap 300, and the stimulation spring 400.

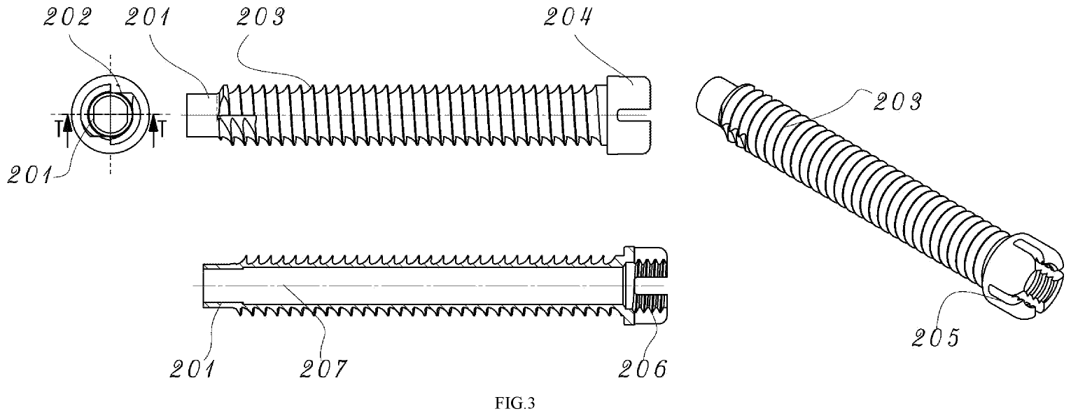

Referring to FIG. 3 presents an isometric view and the corresponding side and sectional views of the female component showing the double flat 201 on the female shaft. This feature can be located externally or internally. When the feature 201 is located internally it can be partial or all along the hollow 207. The double flat stabilizes the male component 100 against any rotational instability in the bone.

Referring to FIG. 3 presents the double self-tapping features, 202. The main shaft of the female component 203, is fully threaded on the female shaft. The head of female 204 is protruding to ensure the obstruction of the female on the lateral cortex. The head of the female has a cross-feature, 205, for connection with the insertion and removal instrument. The threaded nest of the end cap, 300, is shown in FIG.3 feature 206.

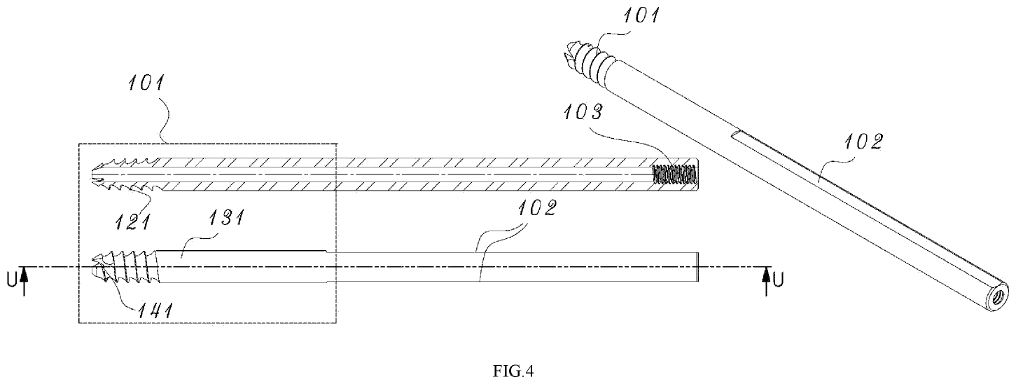

Referring to FIG. 4 presents an isometric view and the corresponding side and sectional views of the male showing the head male component with its sub-features. The head thread 121 allows the screw assembly to be fixed on the femoral head. The male head has a smooth shaft, 131, which of the diameter is slightly larger than the diameter of the thread. The threaded head has double self-tapping features, 141 that allow easy insertion of the male component.

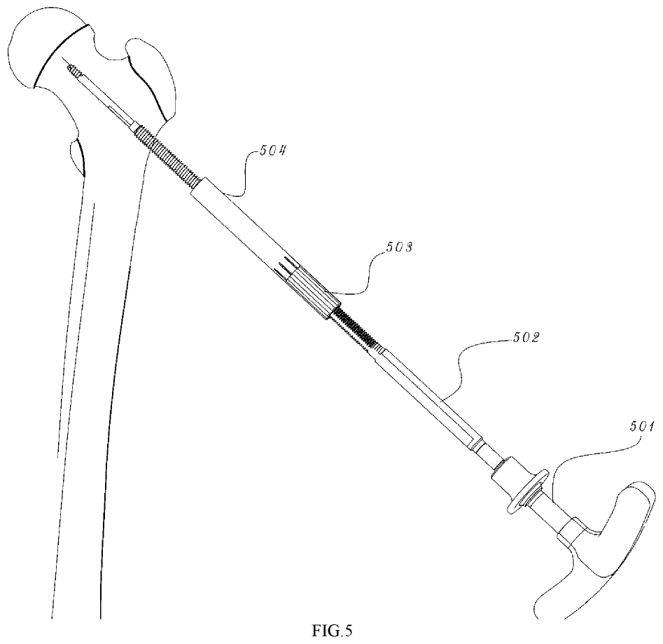

Referring to FIG. 5 presents an isometric view of the adjustable inserter,500 with its four-principal component, the standard T-handle 501, the threaded shaft 502, the adjustment nut 503, and the connector 504. The standard T-handle can be detachable for standardization purpose or solidified with the shaft 502

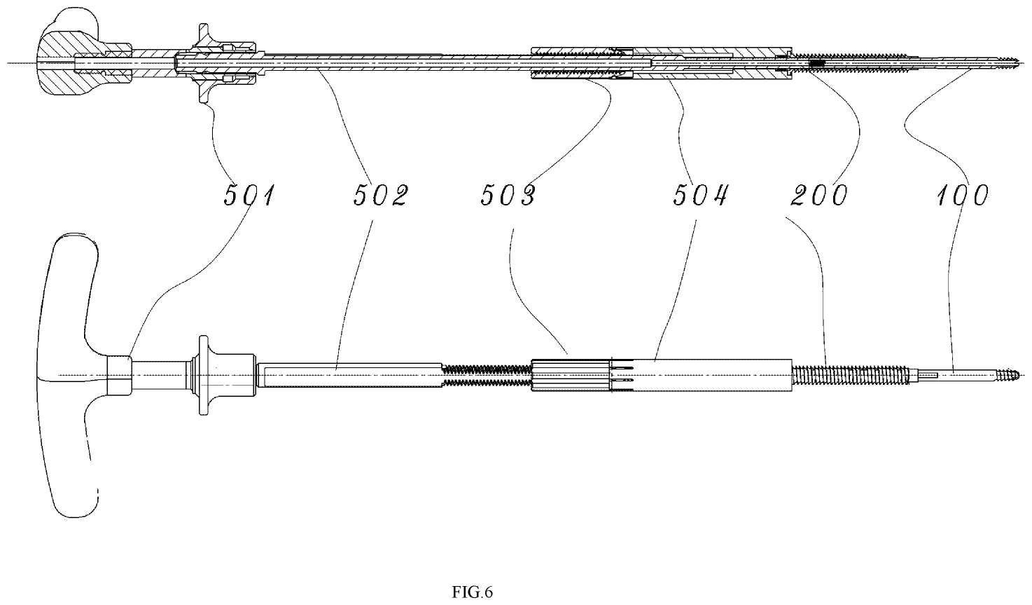

Referring to FIG. 6 presents a side and a section view of the adjustable inserter, once the desired length of the screw is selected the adjustment is obtained by rotating clockwise the nut that advances the connect to a given position. Once the desired position is reached it is maintained in place through a direct contact between the shaft 501 and the male proximal end.

While particular embodiments of the invention were illustrated and depicted, various modifications are possible without dissolution of the invention's spirit and scope as will be obvious to those skilled in the art. Such changes and modifications are within the scope and teachings of this invention as defined in the claims appended hereto.

What is claimed is:

1. An adjustable pediatric hip screw system of two components wherein a male component is nested in a female for the purpose of gliding without rotating and the total length is adjustable to the child's bone anatomy through an adjustment inserter, the assembled screw can nest a simulation spring intended to kindle the physis healing, this spring is framed between the end cap and male component.

2. An insertion instrument that has a threaded shaft, a nut and connector and that is used to adjust the length of the screw assembly in claim 1.

3. The screw assembly of claim 1, wherein the female component is hollow and has an external thread.

4. The screw assembly of claim 1, wherein the female has a double flat can be located externally distally, internally distally, partially, or all along.

5. The screw assembly of claim 1, wherein the double flat of the female component is internal or external and in both cases is partial.

6. The screw assembly of claim 1, wherein the double flat of the female component, if external, is obtained by a special burnishing or any plastic deformation operation that allows a controlled deformation.

7. The screw assembly of claim 1, wherein the head of the female component is cross-shaped for easy bone insertion.

8. The screw assembly of claim 1, wherein the head of the female component has a thread to nest an end cap.

9. The screw nail assembly of claim 1, wherein the female head has two self-tapping features that help to ease the insertion step.

10. The screw assembly of claim 1, wherein the male has a shaft with double flat and cannulated all along.

11. The screw assembly of claim 1, wherein the male has a shaft with a distally threaded head.

12. The screw assembly of claim 1, wherein the male threaded head has a smooth portion that is larger than the thread diameter and acts as a plug against cellular migration from the femoral head to the femoral neck through the physis.

13. The screw assembly of claim 1, wherein the male threaded head has two self-tapping features for easy insertion.

14. The adjustment screw driver of claim 1, wherein the connector does not detach from the and provides a minimum accuracy of 0.1 mm for length adjustment.
